(19) United States
(12) Patent Application Publication
(10) Pub. No.: US 2026/0108505 A1
(43) Pub. Date: Apr. 23, 2026

(54) EYE DROP COMPOSITION COMPRISING NOVEL MOLECULAR ASSOCIATION OF AXITINIB AND METHOD FOR PREPARING THE SAME

(71) Applicant: SCAI THERAPEUTICS CO., LTD., Seoul (KR)

(72) Inventors: Chul Hwan KIM, Daejeon (KR); Kyoung Hee KIM, Daejeon (KR); Sung-Woo HWANG, Busan (KR)

(73) Assignee: SCAI THERAPEUTICS CO., LTD., Seoul (KR)

(21) Appl. No.: 19/147,858

(22) PCT Filed: Mar. 4, 2025
(86) PCT No.: PCT/KR2025/099513

§ 371 Date: Jul. 14, 2025

(30) Foreign Application Priority Data
Apr. 30, 2024 (KR) 10-2024-0057653

(51) Int. Cl. A61K 31/4439; A61K 9/08; A61P 27/02

(52) U.S. Cl. CPC A61K 31/4439; A61K 9/08; A61P 27/02

(57) ABSTRACT

A composition containing a molecular association in which axitinib is physically bound and a method for preparing the composition are disclosed. The composition contains a molecular association in which axitinib is physically bound, a solubilizer, a stabilizer, and an additive. The additive may include any one or more selected from a buffer, an osmotic pressure regulator, and a pH adjuster.

### TECHNICAL FIELD

The present invention relates to a composition comprising a novel molecular association of axitinib, and more specifically, provides an eye drop composition having an excellent effect in treating and preventing wet macular degeneration, by preparing a composition comprising a novel molecular association by a bottom-up method using shear stress.

### BACKGROUND ART

Axitinib is known as an oral selective inhibitor of vascular endothelial growth factor (VEGF) receptors 1, 2 and 3, which is used in the treatment of metastatic renal cell cancer (mRCC). In addition, it is also used as a drug that blocks VEGF signals as a therapeutic agent for wet macular degeneration. EYELEA® from Regeneron Pharmaceuticals, which a product injected into the eye, is known as a therapeutic agent for wet macular degeneration, but products injected into the eye have side effects such as bleeding at the injection site, eye pain, cataracts, vitreous detachment, vitreous floaters and increased intraocular pressure.

Recently, as a new treatment for wet macular degeneration, research is ongoing to develop an eye drop composition using cyclodextrin, which may be expected to deliver the drug to the posterior chamber of the eye to lower retinal cholesterol. It is believed that cyclodextrins are economical, safe, and may provide an efficient route to reduce the effects of retinal aging. However, when the content of cyclodextrin was too high, there were problems in animal models, such as swollen upper and lower eyelids, partial congestion of the eye and nictitating membrane edema. In addition, when the concentration of cyclodextrin is too low compared to the active substance, there is a disadvantage in that the stability of the composition is reduced.

Accordingly, there has been a need for a novel molecular association of axitinib and an eye drop composition comprising the same, having excellent effects in treating and preventing wet macular degeneration by preparing the composition having an easy preparation method and stability.
(Patent Document 1) U.S. Pat. No. 9,968,603 B2

### DISCLOSURE

### Technical Problem

It is an object of the present invention to provide an eye drop composition having an effect of treating wet macular degeneration in vivo. More specifically, it is an object of the present invention to provide an eye drop composition having an excellent therapeutic effect on wet macular degeneration by preparing the composition having an easy preparation method and stability.

### Technical Solution

In order to achieve the above objects,
the present invention provides a composition characterized is by comprising a molecular association in which axitinib physically bound; a solubilizer; a stabilizer; and an additive.

In addition, the present invention provides a method for preparing a composition, comprising the steps of: (a) adding an acid to a molecular association in which axitinib is physically bound to dissolve; (b) adding a solubilizer to the solution and stirring to prepare a first solution; (c) solubilizing a stabilizer in water to prepare a second solution; (d) mixing the first solution and the second solution; (e) adding an additive including a buffer and an osmotic pressure regulator as an active ingredient to the solution prepared in the step (d), adjusting the pH to be 6 to 8 with a pH adjuster, and then adding water to mark it; and (f) sterilizing the solution prepared in the step (e).

### Advantageous Effects

The eye drop composition comprising the novel molecular association of axitinib according to the present invention is a composition having an easy preparation method and stability, and has an excellent therapeutic effect on wet macular degeneration by delivering the active substance well to the posterior chamber of the eye in the form of eye drops, and not causing problems such as swollen upper and lower eyelids, partial congestion of the eye and nictitating membrane edema, which occur when combinations of existing known compositions fail to show effects in vivo.

### DESCRIPTION OF DRAWINGS

FIG. 1 is a graph showing the results of an in vivo drug efficacy evaluation of an exemplary composition of the present invention, showing the results of reducing neovascular area and spot number in a laser-induced monkey model.

FIG. 2 is a graph showing the results of an in vivo drug efficacy evaluation of an exemplary composition of the present invention, showing the results of reducing retinal lesion thickness in a laser-induced monkey model.

### BEST MODE

The present invention provides a composition comprising a molecular association in which axitinib is physically bound. In addition, it provides a composition that may ultimately be used as an eye drop composition and may be used for preventing or treating wet macular degeneration.

Hereinafter, the present invention will be described in more detail.

The composition according to the present invention comprises a molecular association in which axitinib is physically bound; a solubilizer; and a stabilizer.

The composition according to the present invention comprises a molecular association in which axitinib is physically bound.

The molecular association in which axitinib is physically bound may be a molecular association in which axitinib, which is a compound of following Formula (1), is physically bound.

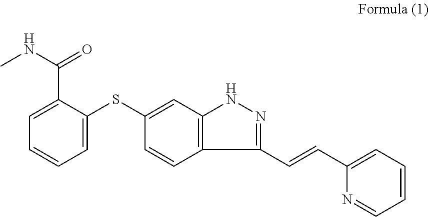 [SMILES: CNC(=O)c1ccccc1Sc1ccc2c(/C=C/c3ccccn3)n[nH]c2c1]

The X-ray powder diffraction spectrum of the molecular association in which axitinib is physically bound has X-ray diffraction peaks at diffraction angles 2θ of 24.99°±0.1° and 26.32°±0.1°.

In addition, the molecular association in which axitinib is physically bound may have a differential scanning calorimetry (DSC) profile with a glass transition at a single endothermic temperature of 220.4±2.0° C. when measured under DSC conditions of 10° C./min heating rate, 99.999% N₂ and 30 to 250° C. That is, while conventional general axitinib exhibits a glass transition at two endothermic temperatures of about 212.5° C. and about 220.6° C. based on the DSC profile, the molecular association in which axitinib is physically bound according to the present invention has a difference in that it has a DSC profile characterized by a glass transition at a single endothermic temperature of 220.4±2.0° C.

In addition, the crystals of the molecular association in which axitinib is physically bound may have an average particle diameter of 2.0 to 15 μm, preferably 3.0 μm or more, 5.0 μm or more, and 13.0 μm or less, 10.0 μm or less. When the crystals of the molecular association have an average particle diameter of exceeding 15.0 μm, there are problems that dispersibility is poor and transparency and permeability are poor. In addition, when the crystals of the molecular association have an average particle diameter of less than 2.0 μm, there are problems that it is difficult to prepare and performance is not achieved.

In addition, the molecular association in which axitinib is physically bound may have an aspect ratio value of 0.3 to 1.0. That is, unlike conventional general axitinibs that have an aspect ratio value of less than 0.3 and thus have an elongated rod shape, the molecular association in which axitinib is physically bound according to the present invention has a physically bound structure of pure axitinib, and thus has a difference in that it exhibits the characteristic of having an aspect ratio value of 0.3 or more, and has a relatively round shape.

The molecular association in which axitinib is physically bound may have an aspect ratio of 0.3 or more, 0.4 or more, 0.5 or more, 0.6 or more, 0.7 or more, 0.8 or more, 0.9 or more, and may have an aspect ratio of 1.0 or less, 0.9 or less, 0.8 or less.

In the present invention, the aspect ratio of a particle may be determined by measuring the length and thickness of the particle using any suitable measurement technique, preferably using a dynamic image analysis method performed according to the ISO 13322-2:2006 standard and calculating the aspect ratio from the measured dimensions of the particle as described above.

In addition, the molecular association in which axitinib is physically bound may have the following solubility: a dissolution concentration at pH 1 of 3.0 mg/mL or more and a dissolution concentration at pH 2 of 0.1 mg/mL or more.

Specifically, the molecular association in which axitinib is physically bound may have a dissolution concentration at pH 1 of 3.0 mg/mL or more, 3.5 mg/mL or more, 4.0 mg/mL or more, 4.3 mg/mL or more, and the upper limit may be, but is not particularly limited to, 10.0 mg/mL or less.

In addition, specifically, the molecular association in which axitinib is physically bound may have a dissolution concentration at pH 2 of 0.1 mg/mL or more, 0.3 mg/mL or more, 0.5 mg/mL or more, 1.0 mg/mL or more, 1.5 mg/mL or more, 1.7 mg/mL or more, and the upper limit may be, but is not particularly limited to, 5.0 mg/mL or less.

The molecular association in which axitinib is physically bound may have a solubility that is 1.5 or 2 times higher than the solubility of the native axitinib itself.

The molecular association in which axitinib is physically bound may be prepared by applying shear stress to a solution containing axitinib or a salt of axitinib, which is a precursor of the structure.

The shear stress applied to the solution containing axitinib, which is a precursor of the structure, may be either mechanical shear stress or ultrasonic application.

The mechanical shear stress may be applied by passing the solution through a column filled with silica or a filter paper. Hereinafter, mechanical shear stress will be described in detail.

According to an embodiment of the present invention, the mechanical shear stress may be applied by passing a solution containing axitinib through a column filled with silica. When the solution containing axitinib passes through a column filled with silica or the like, the axitinib undergoes a very high shear stress as it passes through a physically narrow area.

The silica may be spherical or polygonal, but its shape is not limited.

The size of the silica may be 0.01 to 100 μm, preferably 0.1 to 10 μm, and more preferably 2.5 to 3.7 μm. When the size of the silica is less than 0.01 μm or exceeds 100 μm, even if the solution containing axitinib passes through a column filled with silica, shear stress may not be applied, so that there may be no change in the structure.

A negative pressure of 0.1 bar to 1.0 bar or 0.2 bar to 0.9 bar may be applied to the bottom of the column filled with the silica. When the negative pressure applied to the bottom of the column filled with the silica is less than 0.1 bar, the time required for the solution containing axitinib to pass through the column is increased, so that the production time of axitinib according to the present invention may be delayed. In addition, when the negative pressure applied to the bottom of the column filled with the silica exceeds 1.0 bar, the time required for the solution containing axitinib to pass through the column is reduced, so that the production time of axitinib according to the present invention may be shortened, but production costs may increase because additional pump equipment is required.

According to another embodiment of the present invention, the mechanical shear stress may be applied by passing the solution containing axitinib through one or more filter papers. When passing through the one or more filter papers, the axitinib undergoes a very high shear stress as it passes through a physically narrow area.

The filter paper may be one filter paper or two or more filter papers. When the filter paper consists of two or more filter papers, the filter papers may be arranged in a stacked manner. When the filter paper consists of two or more filter papers, high shear stress may be provided compared to a single filter paper.

The pore size of the filter paper may be 0.1 to 5.0 microns or 0.3 to 4.5 microns. When the pore size of the filter paper is less than 0.1 micron, the amount of the solution containing axitinib passing through or filtering through the filter paper is too small, so that the production speed of axitinib according to the present invention may be reduced, and when the pore size of the filter paper exceeds 5.0 microns, the solution containing axitinib simply passes through the filter paper, so that shear stress may not be effectively applied.

The composition according to the present invention comprises a solubilizer.

In the present invention, a solubilizer is used to increase the solubility of a drug, and the solubilizer used in the present invention may include any one or more selected from the group consisting of alphacyclodextrin (alpha-CD), beta-cyclodextrin (beta-CD), gamma-cyclodextrin (gamma-CD), 2-hydroxypropyl-beta-cyclodextrin (HP-beta-CD), sulfobutylether beta-cyclodextrin (SBE-beta-CD); 2-hydroxypropyl-alpha-cyclodextrin; randomly methylated beta-cyclodextrin; 2-O-methyl-beta-cyclodextrin; 2,6-di-O-methyl-beta-cyclodextrin; heptakis(2,3,6-tri-O-methyl)-beta-cyclodextrin; carboxymethyl-beta-cyclodextrin; carboxyethyl-beta-cyclodextrin; hydroxyethyl-beta-cyclodextrin; maltosyl-beta-cyclodextrin; 3,6-(N,N,N-trimethylammonium)propyl-beta-cyclodextrin; acetyl-beta-cyclodextrin; 2,6-di-O-methyl-gamma-cyclodextrin; 2-hydroxypropyl-gamma-cyclodextrin; or sulfobutylether gamma-cyclodextrin; optionally, wherein the cyclodextrin is alpha-cyclodextrin (alpha-CD), beta-cyclodextrin (beta-CD), gamma-cyclodextrin (gamma-CD), 2-hydroxypropyl-beta-cyclodextrin (HP-beta-CD) or sulfobutylated-beta-cyclodextrin (SBE-beta-CD), and cyclodextrin derivatives.

The solubilizer is a cyclodextrin derivative, and when existing cyclodextrins are used as a component of a composition, they have been used in a form in which they are complexed with a drug. However, in the present invention, cyclodextrin and cyclodextrin derivatives as components of the composition may be used for simple solubilization of drugs rather than in the form of a complex.

The composition according to the present invention comprises a stabilizer.

In the present invention, the stabilizer is used to maintain the stability of the drug and preserve the efficacy, and may exist in various forms, and may be used to increase the solubility of the selected drug depending on the characteristics and use of the drug, and the stabilizer according to the present invention may include any one or more selected from the group consisting of methylcellulose (MC), hydroxymethyl cellulose (HMC), carboxymethylcellulose (CMC), hydroxypropyl methylcellulose (HPMC), hydroxyethyl cellulose (HEC), and cellulose derivatives.

The composition according to the present invention may further comprise an additive, and the additive may be used without any particular limitation as long as it is generally included, and preferably, an additive comprising any one or more selected from the group consisting of a buffer, an osmotic pressure regulator and a pH adjuster may be used.

The buffer serves to maintain the stability and efficacy of the drug by maintaining a biocompatible pH (hydrogen ion concentration) range at a certain level, and specifically, the composition according to the present invention may comprise any one or more selected from the group consisting of citric acid, phosphoric acid, boric acid, tartaric acid, acetic acid and amino acids as the buffer.

The osmotic pressure regulator serves to maintain the appropriate melting point (dissolution point) of the drug and to control the size and dispersion of drug particles to help better absorption when administered, and specifically, the composition according to the present invention may comprise any one or more selected from the group consisting of sorbitol, mannitol, dextrose, sucrose and glycerin as the osmotic pressure regulator.

The pH adjuster serves to provide an appropriate chemical environment by adjusting the pH value of the drug, and specifically, the composition according to the present invention may use an alkalizing agent as the pH adjuster.

The composition according to the present invention comprises a molecular association in which axitinib is physically bound; a solubilizer; and a stabilizer,
wherein the composition may comprise 0.02 to 0.12 w/v % of the molecular association in which axitinib is physically bound; 2 to 12 w/v % of the solubilizer; and 0.01 to 1.0 w/v % of the stabilizer, based on the entire composition. When the content of the molecular association in which axitinib is physically bound is lower than 0.02 w/v %, the effect of using the drug may be minimal, and when the content is higher than 0.12 w/v %, a problem such as swollen upper and lower eyelids may occur.

The composition according to the present invention may further comprise an additive, wherein the additive may include a buffer and an osmotic pressure regulator, and in this case, based on the entire composition, the buffer may comprise 0.5 to 1.5 w/v %; and the osmotic pressure regulator may comprise 0.1 to 5 w/v %.

The composition according to the present invention may comprise a pH adjuster as an additive, and the pH may be adjusted to be 6 to 8 using the pH adjuster.

The composition according to the present invention may be for eye drops.

The composition according to the present invention may have an appropriate viscosity for use as an eye drop, and a specific viscosity may be 100 m·Pas or less, 80 m·Pas or less, 60 m·Pas or less, or 40 m·Pas or less. The lower limit of the viscosity is also not particularly limited, but may typically be 5 m·Pas or more. When the viscosity of the eye drop composition exceeds 100 m·Pas, it may be very sticky and thus the feeling of eye drop may be reduced.

The composition according to the present invention may be a pharmaceutical composition for preventing or treating wet macular degeneration.

The macula is located at the center of the retina and plays an important role in vision, and the wet macular degeneration disease is a disease in which the macular area in the center of the retina is damaged due to aging, and is mainly found in patients with eyelid symptoms. The wet macular degeneration disease mainly occurs in the elderly and is one of the most common causes of vision loss.

The macular degeneration is broadly classified into dry macular degeneration and wet macular degeneration. Of these, wet macular degeneration is a condition in which abnormal blood vessels form in the retina or blood vessels are abnormally exposed, and these changes may include blood vessel leakage, neovascularization, the formation of new blood vessels on the retina, etc., and this process may lead to a loss of vision.

The composition according to the present invention may prevent or treat such symptoms of wet macular degeneration.

The present invention provides a method for preparing a composition, comprising the steps of: (a) adding an acid to a molecular association in which axitinib is physically bound to dissolve; (b) adding a solubilizer to the solution and stirring to prepare a first solution; (c) solubilizing a stabilizer in water to prepare a second solution; (d) mixing the first solution and the second solution; (e) adding an additive including a buffer and an osmotic pressure regulator as an active ingredient to the solution prepared in the step (d), adjusting the pH to be 6 to 8 with a pH adjuster, and then adding water to mark it; and (f) sterilizing the solution prepared in the step (e).

In the preparation method, the contents of the molecular association in which axitinib is physically bound, the solubilizer, the stabilizer, the buffer and the osmotic pressure regulator are the same as those described above in the composition.

The characteristics of the preparation method may be specified, but are not limited to, as follows.

In step (a), a solution may be obtained by dissolving or suspending a molecular association in which axitinib is physically bound, in an acidic aqueous solution in which an acidifying agent such as hydrochloric acid has been added to water. In addition, when the acidifying agent is mixed with water, the aqueous solution may be used after cooling to 20 to 30° C. due to a temperature increase.

When a cellulose derivative is used as the stabilizer in step (c), the temperature may be raised to 60° C. or more to solubilize the cellulose derivative, or a device commonly used for making cellulose into an aqueous solution, such as a stirrer or homogenizer, may be used to dissolve by stirring.

The order of the steps (b) and (c) may be changed and used, and it is possible to add the aqueous solution of step (b) to the aqueous solution of step (c) and then proceed to the next step.

When proceeding with step (d), it may be done at a temperature of 30° C. or less, with shading.

Sterilization in step (f) may be performed by aseptic filtration, and sterilization may be performed by filtration through a filter.

Hereinafter, the present invention will be described in more detail through the Examples of the present invention. It will go without saying that the present invention is not limited to these examples.

### PREPARATIVE EXAMPLE

### 1. Preparation of Molecular Association (SCAI-005)

16.0 g of axitinib (Shilpa company, India) was dissolved in 16.0 kg of ethanol (94.5% ethanol, Samchun company) to prepare a dissolved solution of axitinib at a concentration of about 0.1%.

270 g of SYLOID 244FP (GRACE company, USA) was wetted with 4.32 kg of ethanol (94.5% ethanol, Samchun company), and a 1.0 μm paper filter was combined with a Nutsche filter with a diameter of 350 mm. A SYLOID 244FP column with a height of approximately 1.4 cm was prepared by pouring the SYLOID 244FP solution wetted with ethanol into the Nutsche filter.

1.08 kg of the prepared 94.5% ethanol was added onto the column using vacuum to strengthen the SYLOID 244FP column. The prepared dissolved solution of axitinib was added, and an additional 3.24 kg of 94.5% ethanol was passed through the column to recover the axitinib remaining in SYLOID 244FP. In this case, the weight of the axitinib effluent was approximately 21.86 kg.

The axitinib effluent was filtered using a 0.45 μm PVDF membrane filter and concentrated to a concentrate concentration of 3.0 mg/g using a rotary vacuum concentrator. Once concentration was completed, the axitinib concentrate was filtered using a 0.2 μm PVDF membrane filter.

53.0 kg of purified water was added to a 100 L reactor, and the prepared axitinib concentrate was slowly added while rapidly stirring the purified water. After the addition was completed, additional stirring was performed for 30 minutes. The mixed solution was filtered using a 1.0 μm paper filter.

The filtered cake was vacuum decompressed for 30 minutes and dried using nitrogen for 2 hours. In addition, it was dried in a vacuum oven at 25° C. for 38 hours to obtain 14.07 g (yield of 88%) of axitinib as a white powder.

### Preparative Experimental Example 1. X-Ray Diffractometer (XRD) of Novel Axitinib Polymorph (SCAI-Form)

The reagent was placed in a sample holder, pressed with a glass rod, and filling molded into the filling part, and it shows a crystal form when tested according to the X-ray powder diffraction method among the general test methods in the “Korean Pharmacopoeia”.

### Operating Conditions

TABLE 1

| | Actual measuring | | | Actual measuring |
|---|---|---|---|---|
| Conditions | conditions | | Conditions | conditions |
| X-ray tube | Copper K2 a | | 2 Theta start | 5° |
| Counter | Scintillation | | 2 theta end | 60° |
| | counter | | | |
| Voltage | 40 | kV | Speed | 2°/min |
| Current | 15 | mA | Scane type | Vertical type |
| Wavelength | 1.5406 | Å | Scane mode | continuous |

| | | | |
|---|---|---|---|
| Detector | D/tex Ultra2 | Divergence slit | 1.25° DS/21HS |

X-ray powder diffraction patterns of various polymorphic forms were performed on a Rigaku Miniflex600 using copper radiation (CuKα, wavelength of 1.5406 Å). The voltage and current of the tube were set to 40 kV and 15 mA, respectively. The divergence and scattering slits were set to 8.0 mm and the receiving slit was set to 13.0 mm. Diffracted radiation was detected with D/tex Ultra2. Theta-2 theta continuous scan was used at 2.0°/min (1 sec/0.03° step) from 3.0° to 60° 2θ. Alumina standards were analyzed to confirm instrument alignment. Data was collected and analyzed using SmartLab Studio II.

X-ray powder diffraction patterns were measured on a Rigaku Miniflex 600 using copper radiation (CuKα, wavelength of 1.5406 Å). The voltage and current of the tube were set to 40 kV and 15 mA, respectively. The divergence and scattering slits were set to 8.0 mm and the receiving slit was set to 13.0 mm. Diffracted radiation was detected with D/tex Ultra2. Theta-2 theta continuous scan was used at 2.0°/min (1 sec/0.03°) from 3.0° to 60° 2θ. Alumina standards were analyzed to confirm instrument alignment. Data was collected and analyzed using SmartLab Studio II.

2 Theta and Relative Intensity, which are the XRD Results of API, SCAI-Form

TABLE 2

| API | | SCAI-Form (AXTEN03A) | |
|---|---|---|---|
| Angle | Relative intensity (%) | Angle | Relative intensity (%) |
| 8.19 | 102.70 | 8.84 | 53.2 |
| 11.92 | 4.23 | 11.99 | 133.1 |
| 14.74 | 3.93 | 14.60 | 26 |
| 15.34 | 20.13 | 15.24 | 51.9 |
| 15.56 | 43.41 | 15.70 | 76.6 |
| 17.45 | 57.19 | 17.71 | 27.6 |
| 19.39 | 4.72 | 19.33 | 58.1 |
| 20.69 | 8.41 | 20.65 | 58.8 |
| 21.40 | 9.21 | 21.70 | 85.1 |
| 23.29 | 44.46 | 23.23 | 53.4 |
| 24.01 | 55.39 | 24.19 | 38 |
| 25.96 | 100 | 24.99 (25.0) | 100 |
| 26.24 | 3.75 | 26.32 (26.3) | 38.8 |
| 27.78 | 8.13 | 27.59 | 11.9 |

### Preparative Experimental Example 2. Differential Scanning Calorimetry (DSC) of Novel Axitinib Polymorph (SCAI-Form)

It was measured using a heating program [Table 3] in a differential scanning calorimeter device. In this case, the sample amount is recommended to be 4.0 mg or less, the environment within the device is maintained as nitrogen, and the nitrogen flow rate is 10 mL/min.

TABLE 3

| | | | |
|---|---|---|---|
| Rate | Target | Hold | Record |

| | | | | | |
|---|---|---|---|---|---|
| 20° C./min | 100° | C. | 20 | min | |
| 10° C./min | 30° | C. | 5 | min | — |
| 10° C./min | 250° | C. | 5 | min | ✓ |

### Peak Temperature and ΔH of API and SCAI-Form

TABLE 4

| | API | SCAI-Form (AXTEN03A) |
|---|---|---|
| Peak temp. (° C.) | 212.5 | 220.4 |
| | 220.6 | |
| ΔH (J/g) | 129.1 | 131.7 |

### Preparative Experimental Example 3. Scanning Electron Microscope (SEM) of Novel Axitinib Polymorph (SCAI-Form)

### Measuring Conditions

Powder samples were placed on carbon tape fixed to aluminum stubs. The samples were scanned in FE-SEM using JSM-IT800, Jeol. Images were acquired at an acceleration voltage of 1.00 kV using a secondary electron detector.

The crystal structures of Axitinib API and SCAI-Form were analyzed using the above measurement method. As shown in Table 5 below, it could be confirmed that the crystals of the molecular association had an average particle size of 2 to 15 μm.

### Particle Diameter

TABLE 5

| Classification | Diameter (length, μm) |
|---|---|
| Average value | 6.68 |
| Minimum value | 3.29 |
| Maximum value | 12.84 |

### Preparative Experimental Example 4. Solubility of New Axitinib Polymorph (SCAI-Form)

Method for measuring solubility at pH 1: Stirred for 15 minutes at a concentration of 5 mg/mL, and then filtered, diluted 10 times with DW, and analyzed. (The value calculated as [measured value×10] is described in the results)

Method for measuring solubility at pH 2: Stirred for 15 minutes at a concentration of 1 mg/mL, and then filtered, and analyzed.

### Solubility

TABLE 6

| | | Dissolution | Dissolution |
|---|---|---|---|
| | | concentration | concentration |
| Classification | Batch No. | at pH 1 (mg/mL) | at pH 2 (mg/mL) |
| API | Axitinib API | 2.661 | 0.073 |
| | (Shilpa) | | |
| SCAI-Form | AXTEN03A | 4.363 | 0.171 |

From the above, it can be seen that the solubility of SCAI-Form is about twice higher than that of API.

### EXAMPLES

### 1. Preparation Method and Composition of Eye Drops Comprising SCAI-005

Eye drops of Examples 1 to 15 were prepared by the following method under the conditions of Tables 7 and 8 below.

1) After hydrochloric acid was added to water and SCAI-005 was dissolved, hydroxypropyl beta-cyclodextrin (HP-beta-CD) or hydroxypropyl gamma-cyclodextrin (HP-gamma-CD) was added hereto as a solubilizer and stirred.

2) A stabilizer was dissolved in water.

3) The solutions (1) and (2) were mixed.

4) An additive (buffer, osmotic pressure regulator) as an active ingredient was added to the solution (3), and the pH was adjusted to be about 7 with an alkalizing agent, and then water was added thereto to be marked.

TABLE 7

| | Example | Example | Example | Example | Example | Example | Example | Example |
|---|---|---|---|---|---|---|---|---|
| | 1 | 2 | 3 | 4 | 5 | 6 | 7 | 8 |
| Batch No. | (JE30)- | (JE30)- | (JE30)- | (JE30)- | (JE30)- | (JE82- | (JE82- | (JE91A)-100 |
| | 03 | 27 | 29 | 30 | 30-1 | 6)-66 | 6)-72 | |
| SCAI-005 | 0.02% | | | | | | | |
| HP-β- | 1% | 1% | 1% | 1% | | 2% | 2.4% | 2.3% |
| Cyclodextrin | | | | | | | | |
| HP-γ- | — | — | | | 1% | | | |
| Cyclodextrin | | | | | | | | |
| HPMC 2910 | — | 0.1% | | 0.1% | 0.1 | 0.1 | 0.1 | 0.1 |
| (4000 mPas) | | | | | | | | |
| Povidone K30 | 1% | 1% | 2.5% | — | — | | | |
| Povidone K90 | — | — | — | — | — | | | |

| | |
|---|---|
| Citric buffer | 0.728% |
| 1N HCl | Adequate amount |
| 1N NaOH | Adequate amount |

TABLE 8

| | Example | Example | Example | Example | Example | Example | Example |
|---|---|---|---|---|---|---|---|
| | 9 | 10 | 11 | 12 | 13 | 14 | 15 |
| Batch No. | (JE91A)- | (JE91A)- | (JE91A)- | (JE91A)- | (JE91A)- | (JE88)- | (JE91A)- |
| | 101 | 110 | 102 | 103 | 112 | 94 | 104 |
| SCAI-005 | 0.02% | 0.04% | | | 0.08% | | |
| HP-β- | 2.8% | 4% | 5.2% | 5.6% | 7% | 8% | 9.6%- |
| Cyclodextrin | | | | | | | |
| HPMC 2910 | 0.1% | | | | | | |
| (4000 mPas) | | | | | | | |

| | |
|---|---|
| Citric buffer | 0.728% |
| 1N HCl | Adequate amount |
| 1N NaOH | Adequate amount |

### EXPERIMENTAL EXAMPLES

The following experiments were conducted on the eye drop compositions of Examples 1 to 15 prepared above.

### Experimental Example 1. Stability Test (Accelerated Conditions)

When an accelerated test is conducted according to the Korean Pharmacopoeia, it must be stable for up to 6 months depending on the accelerated test conditions. After Examples 1 to 15 were prepared, the property was visually confirmed according to an accelerated test, and the change in content was measured using liquid chromatography, and then the results are shown in Table 9.

TABLE 9

| | | | 1 Week | | 3 Months | | 6 Months | |
|---|---|---|---|---|---|---|---|---|
| | | | accelerated test | | accelerated test | | accelerated test | |
| | | | (40 ± 2° C., 25% | | (40 ± 2° C., 25% | | (40 ± 2° C., 25% | |
| | Initial | | RH or less) | | RH or less) | | RH or less) | |
| | Content | Property | Content | Property | Content | Property | Content | Property |
| Example 1 | 99.09% | Clear, | 27.80% | Precipi- | — | — | — | — |
| | | Colorless | | tated | | | | |
| Example 2 | 102.31% | Clear, | 75.72% | Precipi- | — | — | — | — |
| | | Colorless | | tated | | | | |
| Example 3 | 102.35% | Clear, | 73.79% | Precipi- | — | — | — | — |
| | | Colorless | | tated | | | | |
| Example 4 | 101.04% | Clear, | 81.30% | Precipi- | — | — | — | — |
| | | Colorless | | tated | | | | |
| Example 5 | — | Precipi- | — | — | — | — | — | — |
| | | tated | | | | | | |
| Example 6 | 101.64% | Clear, | 100.90% | Clear, | Precipi- | — | — | — |
| | | Colorless | | Colorless | tated | | | |
| Example 7 | 100.56% | Clear, | 100.52% | Clear, | 75.68% | Precipi- | — | — |
| | | Colorless | | Colorless | | tated | | |
| Example 8 | 100.45% | Clear, | — | Clear, | 101.90% | Clear, | — | Precipi- |
| | | Colorless | | Colorless | | Colorless | | tated |
| Example 9 | 101.26% | Clear, | — | Clear, | 102.98% | Clear, | 97.27% | Clear, |
| | | Colorless | | Colorless | | Colorless | | Colorless |
| Example 10 | 98.60% | Clear, | — | Clear, | — | Precipi- | — | — |
| | | Colorless | | Colorless | | tated | | |
| Example 11 | 99.90% | Clear, | — | Clear, | 102.03% | Clear, | 98.29% | Precipi- |
| | | Colorless | | Colorless | | Colorless | | tated |
| Example 12 | 99.61% | Clear, | — | Clear, | 101.58% | Clear, | 102.85% | Clear, |
| | | Colorless | | Colorless | | Colorless | | Colorless |
| Example 13 | 100.99% | Clear, | — | Clear, | — | Precipi- | — | — |
| | | Colorless | | Colorless | | tated | | |
| Example 14 | 99.38% | Clear, | — | Clear, | 98.85% | Clear, | 103.27% | Clear, |
| | | Colorless | | Colorless | | Colorless | | Colorless |
| Example 15 | 99.38% | Clear, | — | Clear, | 101.41% | Clear, | 106.05% | Clear, |
| | | Colorless | | Colorless | | Colorless | | Colorless |

As can be seen from Table 9, in Examples 1 to 8, 10, 11 and 13, it was confirmed that the stability was poor under accelerated test conditions, and in the results of Examples 9, 12, 14 and 15, it was confirmed that the amounts of SCAI-005 and hydroxy beta-cyclodextrin were stabilized from certain concentration.

### Experimental Example 2. In-Vivo Pharmacokinetics of SCAI-005

In this experiment, neovascularization was induced by damaging the choroid of both eyes using a laser in a monkey animal model, and then treated with drugs, and the efficacy is compared and evaluated by performing fundus fluorescein angiography (FFA) and optical coherence tomography (OCT) and measuring the neovascular area and spot number in the damaged area and the retinal lesion thickness.

### Laser Photocoagulation and Group Assignment

Experimental CNV in monkey eyes was induced using a laser photocoagulation system (Vitra 532 nm, Quantel Medical, France).

Laser parameters: laser wavelength of 532 nm; spot size of 50 μm; energy of 600-800 mW; exposure time of 0.1 s.

A total of eight rhesus monkeys were used to induce CNV. The animals were anesthetized by intramuscular injection of ketamine hydrochloride (20 mg/kg) and dexmedetomidine hydrochloride (0.03 mg/kg), and then both eyes of each monkey were burned at 8-9 points around the macula, approximately one disc diameter from the macula. Care was taken not to aim the laser at the macula. The force used depended on the morphology of the subretinal bleb indicating a perforation of the Bruch's membrane. If no bled formed, higher energy was used at the next point. Color fundus photography (Retinal Camera TRC-50DX, Topcon Healthcare, Japan) was performed immediately after laser photocoagulation to observe the laser dots. Fundus fluorescein angiography (FFA) examination was performed 12 days after laser induction. Modeling was assessed as successful if there was one or more fluorescence leakage point of grade III and/or grade IV. Animals were enrolled in treatment and randomly divided into two groups based on the total leakage area of grade III and IV laser spots (Tables 10 and 11). The monkeys that were not included were returned to the PRIMED Animal Care Department.

TABLE 10

| | | Amount | | | | |
|---|---|---|---|---|---|---|
| | | of | | | | |
| | Test | monkey | | Dose | | Route of |
| Group | Article | (eyes) | Dosage | volume | Frequency | Dosing |
| Vehicle | Vehicle | 3 (6) | 1 drop | 50 | 4 times/day | Topical |
| | | | | μL/drop | for 28 days | administration |
| Test | Example 15: | 3 (6) | 1 drop | 50 | 4 times/day | Topical |
| article | SCAI-005 | | | μL/drop | for 28 days | administration |
| | (0.08%) | | | | | |
| | Ophthalmic | | | | | |
| | solution | | | | | |

### Grouping

TABLE 11

| | | Storage | Physical | Concen- |
|---|---|---|---|---|
| Article | Lot Number | condition | properties | tration |
| Example 15: | AXTSW(JE91A)- | 2-8° C. | Clear, | Axitinib |
| SCAI-005 | 107 | | Colorless | 0.08% |
| (0.08%) | | | solution | (0.8 mg/ml) |
| Ophthalmic | | | | |
| solution | | | | |
| Vehicle | AXTSW(00)- | 2-8° C. | Clear, | — |
| | 018 | | Colorless | |
| | | | solution | |

### Evaluation Procedures

### Anesthesia and Pupil Dilation

Before acquiring images, animals were anesthetized by intramuscular injection of ketamine hydrochloride (20 mg/kg) and dexmedetomidine hydrochloride (0.03 mg/kg). To dilate the pupil, two drops of tropicamide phenylephrine eye drop were instilled into each eye after anesthesia. After anesthesia, the animals were kept in a dark room until the pupil diameter exceeds 6 mm. A spectrum of self-maintaining eyelids was placed on the eyes.

### Fundus Photography (FP)

Retinal camera: Topcon TRC-50DX. Frequency: a total of 5 times, 1 time before laser photocoagulation, 1 time immediately after laser induction, 1 time before administration (−Day 2), 1 time on Day 14 and 1 time on Day 28 after the first administration.

FP image acquisition protocol: The animal's forehead was supported against a forehead support. Before the image was taken, the working distance was adjusted and focus was achieved. It was confirmed that a central macular image was obtained. Animals were managed according to relevant SOPs during the examination.

### Fundus Fluorescein Angiography (FFA)

Retinal camera: Topcon TRC-50DX.

Frequency: a total of 4 times, 1 time before laser photocoagulation, 1 time before administration (−Day 2), 1 time on Day 14 and 1 time on Day 28 after the first administration.

FFA image acquisition protocol: The FP images were taken, and then FFA was performed. After intravenous infusion of 10% sodium fluorescein (Alcon Laboratories, USA) at a dose of 0.075 mL/kg, rapid posterior polar photographs of the right eye were taken. The left eye was then used to monitor leakage of fluorescein related to the CNV lesion at serial time points (1, 5, and 10 minutes).

To quantify the area of CNV leakage, the total area of hyperfluorescence per eye was measured in late FFA images using Image J software (version 1.52a, Wayne Rasband National Institutes of Health, USA). Images from the later stage (10 min) were also used to calculate the amount and grade of CNV spots (Table 12).

TABLE 12

| Lesion Grade | Definition |
|---|---|
| Grade I | No hyperfluorescence |
| Grade II | Leak-free hyperfluorescence |
| Grade III | Early hyperfluorescence or mild passing |
| | and delayed leakage |
| Grade IV | Passing of bright hyperfluorescence leaking |
| | beyond the burn site border |

Optical coherence tomography (OCT): Heidelberg Spectralis OCT plus.

Frequency: a total of 4 times, 1 time before laser photocoagulation, 1 time before administration (−Day 2), 1 time on Day 14 and 1 time on Day 28 after the first administration.

OCT image acquisition protocol: FP and FFA images were taken, and then OCT scans were performed. Efforts were made to maintain uniformly illuminated and well-focused fundus images and high-quality OCT. Once the focus was aligned on the central macula by observation of the monitor screen, the monkey's eyes were identified by applying a high-speed macular scan process. Retinal thickness was automatically measured using software embedded in the Heidelberg OCT by measuring the distance between the internal border membrane and Bruch's membrane. Two membrane lines were found manually. The location with the maximum thickness around the grade III or IV point was selected for measurements. Heidelberg OCT unique has technical characteristics that ensure longitudinal measurements of retinal thickness at the same location.

### Data Analysis

Data analysis consisted of total area of fluorescein leakage points at one of the registered photocoagulation points, volume of fluorescein leakage points, and retinal thickness. Animal observations, photography, fundus and slit-lamp examinations were recorded and summarized in a report. The mean and standard deviation of the percent change in the total area of fluorescein leakage spots and the percent change in retinal thickness were calculated. Microsoft Office Excel 2013 and SPSS Statistics 13.0 were used for data processing and statistical analysis.

### Results; SCAI-005 (Axitinib) Efficacy Trial in Monkeys

### Relative Leakage Area, Change in Leakage Point

Leakage area of grade 3/4 CNV lesions (mm², Mean±SD)

TABLE 13

Treatment Day

| Group | Baseline | Day 14 | Day 28 |
|---|---|---|---|
| Vehicle | 9.39 ± 1.17 | 11.59 ± 2.62 | 11.25 ± 3.56 |
| (n = 6 eyes) | | | |
| SCAI-005 | 9.01 ± 2.25 | 9.65 ± 4.85 | 6.46 ± 3.30* |
| (n = 6 eyes) | | | |

Note:
(1) Baseline: After induction and before treatment;
(2) Compared with baseline,
*p < 0.05,
**p < 0.01

### Percent Change of Leakage Area (%, Mean±SD)

TABLE 14

Treatment Day

| Group | Day 14 | Day 28 |
|---|---|---|
| Vehicle | 25.2 ± 34.5 | 20.2 ± 36.2 |
| (n = 6 eyes) | | |
| SCAI-005 | 4.5 ± 39.1 | −30.2 ± 28.9* |
| (n = 6 eyes) | | |

Note:
(1) Change of Leakage area = (leakage area after treatment − leakage area at baseline)/leakage area at baseline × 100%;
(2) Compared with vehicle group,
*p < 0.05,
**p < 0.01

### Amount of Grade 3/4 Leakage Spots

TABLE 15

Treatment Day

| Group | baseline | Day 14 | Day 28 |
|---|---|---|---|
| Vehicle | 53 | 53 | 51 |
| (n = 6 eyes) | | | |
| SCAI-005 | 53 | 47* | 43* |
| (n = 6 eyes) | | | |

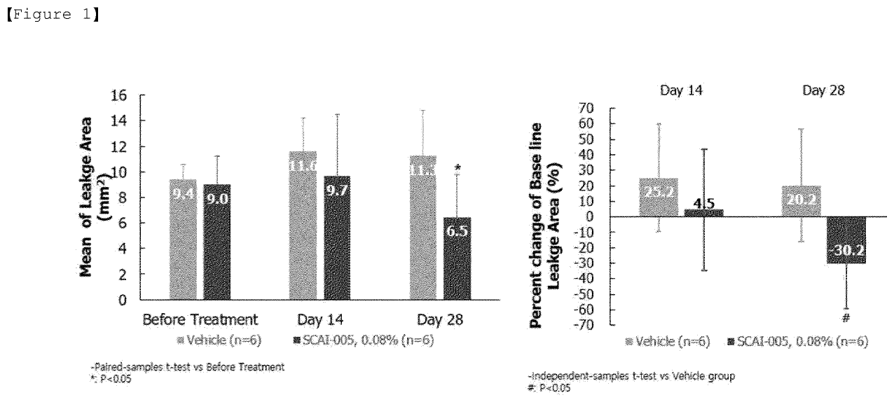

As a result of measurements taken on Day 14 and Day 28 after CNV damage was induced and then the SCAI-005 composition of Example 15 was injected, it was confirmed that the leakage area and the number of grades 3 and 4 (leakage spots) were statistically significantly reduced compared to the control group. (FIG. 1)

### Retinal Thickness (OCT) Changes

Average retinal thickness of grade 3/4 CNV Lesions (μm, Mean±SD)

TABLE 16

Treatment Day

| Group | Baseline | Day 14 | Day 28 |
|---|---|---|---|
| Vehicle | 416 ± 41 | 410 ± 44 | 396 ± 26 |
| (n = 6 eyes) | | | |
| SCAI-005 | 430 ± 45 | 406 ± 68 | 358 ± 40** |
| (n = 6 eyes) | | | |

Note:
(1) Baseline: After induction and before treatment;
(2) Compared with baseline,
*p < 0.05,
**p < 0.01

### Percent Change of Retinal Thickness (%, Mean±SD)

TABLE 17

Treatment Day

| Group | Day 14 | Day 28 |
|---|---|---|
| Vehicle | −5.4 ± 9.5 | −14.1 ± 13.6 |
| (n = 6 eyes) | | |
| SCAI-005 | −26.0 ± 26.3 | −61.4 ± 16.0** |
| (n = 6 eyes) | | |

Note:
(1) Change of retinal thickness = (retinal thickness after treatment − retinal thickness at baseline)/(retinal thickness at baseline − retinal thickness before laser induction) × 100%;
(2) Compared with vehicle group,
*p < 0.05,
**p < 0.01

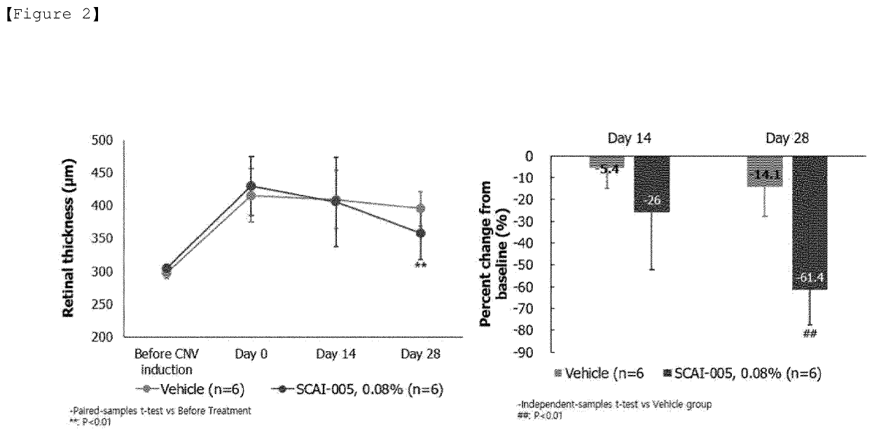

As a result of measurements taken on Day 28 after CNV damage was induced and then the SCAI-005 composition of Example 15 was injected, it was confirmed that the thickness of the retinal lesion was statistically significantly reduced compared to the control group. (FIG. 2)

1. A composition characterized by comprising:
a molecular association in which axitinib is physically bound;
a solubilizer;
a stabilizer; and
an additive,
wherein the molecular association is prepared by applying shear stress by passing a solution containing axitinib through a column filled with silica,
wherein the molecular association has an average particle diameter of 2 to 15 μm,
wherein an X-ray powder diffraction spectrum of the molecular association has X-ray diffraction peaks at diffraction angles 2θ of 24.99°±0.1° and 26.32°±0.1°,
wherein the solubilizer includes any one or more selected from the group consisting of beta-cyclodextrin (beta-CD), 2-hydroxypropyl-beta-cyclodextrin (HP-beta-CD); sulfobutylether beta-cyclodextrin (SBE-beta-CD); randomly methylated beta-cyclodextrin; 2-O-methyl-beta-cyclodextrin; 2,6-di-O-methyl-beta-cyclodextrin; heptakis(2,3,6-tri-O-methyl)-beta-cyclodextrin; carboxymethyl-beta-cyclodextrin; carboxyethyl-beta-cyclodextrin; hydroxyethyl-beta-cyclodextrin; maltosyl-beta-cyclodextrin; 3,6-(N,N,N-trimethylammonium)propyl-beta-cyclodextrin; and acetyl-beta-cyclodextrin,
wherein the stabilizer includes hydroxypropyl methylcellulose (HPMC),
wherein the additive includes any one or more selected from the group consisting of a buffer, an osmotic pressure regulator, and a pH adjuster,
wherein the composition comprises 0.02 to 0.12 w/v % of molecular association based on the entire composition, and
wherein the composition comprises 2 to 12 w/v % of the solubilizer based on a total volume of the composition.

2-4. (canceled)

5. The composition according to claim 1, wherein the buffer includes any one or more selected from the group consisting of citric acid, phosphoric acid, boric acid, tartaric acid, acetic acid, and amino acids.

6. The composition according to claim 1, wherein the osmotic pressure regulator includes any one or more selected from the group consisting of sorbitol, mannitol, dextrose, sucrose, and glycerin.

7. The composition according to claim 1, wherein the pH adjuster is an alkalizing agent.

8. The composition according to claim 1, wherein
the composition comprises 0.01 to 1.0 w/v % of the stabilizer based on the total volume of the composition.

9. The composition according to claim 1, wherein
the composition comprises 0.5 to 1.5 w/v % of the buffer based on the total volume of the composition; and
the composition comprises 0.1 to 5 w/v % of the osmotic pressure regulator based on the total volume of the composition.

10. The composition according to claim 1, wherein a pH of the composition is adjusted to be 6 to 8 using the pH adjuster.

11. (canceled)

12. The composition according to claim 1, wherein the molecular association has a differential scanning calorimetry (DSC) profile with a glass transition at a single endothermic temperature of 220.4±2.0° C. measured under DSC conditions of 10° C./min heating rate, 99.999% N₂ and 30 to 250° C.

13. The composition according to claim 1, wherein the molecular association has an aspect ratio value of 0.3 to 1.0.

14. (canceled)

15. The composition of claim 1 is for eye drops.

16. The composition of claim 1 is a pharmaceutical composition for preventing or treating wet macular degeneration.

17. A method for preparing the composition according to claim 1, comprising the steps of:
(a) adding an acid to a molecular association in which axitinib is physically bound to dissolve;
(b) adding a solubilizer to the solution and stirring to prepare a first solution;
(c) solubilizing a stabilizer in water to prepare a second solution;
(d) mixing the first solution and the second solution;
(e) adding an additive including a buffer and an osmotic pressure regulator as an active ingredient to the solution prepared in the step (d), adjusting the pH to be 6 to 8 with a pH adjuster, and then adding water to mark it; and
(f) sterilizing the solution prepared in the step (e).
